(19) United States
(12) Patent Application Publication
(10) Pub. No.: US 2026/0256953 A1
(43) Pub. Date: Sep. 3, 2026

(54) COMPOSITIONS AND METHODS FOR PREVENTING RETENTION OF PSMA-TARGETED THERAPY

(71) Applicant: CASE WESTERN RESERVE UNIVERSITY, Cleveland, OH (US)

(72) Inventors: Zhenghong Lee, Cleveland, OH (US); James Basilion, Cleveland, OH (US); William Julian, Cleveland, OH (US); Olga Sergeeva, Cleveland, OH (US)

(21) Appl. No.: 18/873,121

(22) PCT Filed: Jun. 9, 2023
(86) PCT No.: PCT/US2023/024926

§ 371 Date: Dec. 9, 2024

Related U.S. Application Data
(60) Provisional application No. 63/350,571, filed on Jun. 9, 2022.
(60) Provisional application No. 63/389,073, filed on Jul. 14, 2022.

(51) Int. Cl. A61K 51/04; A61K 31/381

(52) U.S. Cl. CPC A61K 51/0402; A61K 31/381; A61K 51/0497

(57) ABSTRACT

A method of inhibiting accumulation and/or retention of a prostate-specific membrane antigen (PSMA)-targeted therapy to a non-cancer tissue in a subject includes administering to a subject being treated with the PSMA-targeted therapy a pharmacologically effective amount of an agent that inhibits PSMA-mediated cellular retention of the PSMA-targeted therapy by the non-cancer tissue.

### RELATED APPLICATION

This application claims priority from U.S. Provisional Application Nos. 63/350,571, filed Jun. 9, 2022 and 63/389,073 filed Jul. 14, 2022, the subject matter of which are incorporated herein by reference in their entirety.

### GOVERNMENT FUNDING

This invention was made with government support under CA204373 awarded by the National Institutes of Health. The government has certain rights in the invention.

### BACKGROUND

Recent clinical studies using molecularly targeted radioligand therapy (RLT) have shown remarkable efficacy in cancer treatment. For example, treatment with a short peptide PSMA-targeted ligand, when chelated with an α-emitting radio-metal like Actinium-225 (²²⁵Ac), resulted in rare clinical responses from end-stage patients who had already exhausted approved options. In addition, targeted RLT with ²²⁵Ac for a therapy rescued failed β-emitting radio-metal Lutetium-177 (¹⁷⁷Lu) β-therapy.

While PSMA-targeted ligand treatment is encouraging, patients reported having dry mouth (xerostomia), severe in a number of cases, in addition to dry eyes. PSMA-⁶⁸Ga PET scans show unexpected uptake in all salivary glands (submandibular, sublingual, and parotid) as well as the lacrimal glands.

Existing strategies to reduce the burden of xerostomia have failed to show any efficacy. For targeted RLT-induced xerostomia, suppressing the metabolism of the gland by intraparenchymal injections of botulinum toxin A (Botox), a clinically approved method used in patients who have suffered from sialorrhoea, appeared to be promising, but nonetheless induced temporary xerostomia. External cooling with ice packs was also tested, but did not reduce the uptake of PSMA-targeting radio-ligand by the parotid glands.

### SUMMARY

Embodiments described herein relate to methods of inhibiting accumulation and/or retention of a prostate-specific membrane antigen (PSMA)-targeted therapy in PSMA expressing non-cancer tissue of a subject, and particularly relates to methods of reducing PSMA-targeted radioligand therapy induced salivary gland and/or lacrimal gland damage in a subject receiving PSMA-targeted radioligand therapy for cancer.

In some embodiments, the method can include administering to a subject being treated with the PSMA-targeted therapy a pharmacologically effective amount of an agent that inhibits PSMA-mediated cellular retention of the PSMA-targeted therapy by the non-cancer tissue.

In some embodiments, the agent includes an inhibitor of PSMA-mediated cellular internalization.

In some embodiments, the agent can compete for binding and/or uptake of the PSMA-targeted therapy to PSMA-expressing cancer tissue, PSMA-expressing non-cancerous tumors, and/or PSMA-expressing tumor vasculature in the subject.

In other embodiments, the agent does not compete for binding and/or uptake of the PSMA-targeted therapy to PSMA-expressing cancer tissue, PSMA-expressing non-cancerous tumors, and/or PSMA-expressing tumor vasculature in the subject.

In some embodiments, the agent comprises an inhibitor of clathrin mediated endocytosis (CME). The inhibitor of CME can form a complex with a binding site on a terminal domain of clathrin heavy chain to interfere with CME.

In some embodiments, an inhibitor of CME that can form a complex with a binding site on a terminal domain of clathrin heavy chain can include an aquaporin (AQP) inhibitor, preferably an aquaglyceroporin inhibitor.

In some embodiments, the AQP inhibitor can include at least one of a 1-(5-nitrothiophene-3-yl)urea compound, a 1-(1H-indole-3-yl)urea compound, or a 4-(carbamoylamino)benzoate compound.

In other embodiments, the AQP inhibitor can include at least one of DFP00173, DFP00172, SEW00835, SEW00834, SEW00833, SEW00832, F2740-0111, Z4339273301, HTS13286, 9016645, 9053871, 7791389, or IVK/0050432.

In other embodiments, the inhibitor of CME can include 2-(4-aminobenzyl)-1,3-dioxo-2,3-dihydro-1H-benzo[de]isoquinoline-5-sulfonate (Pitstop 1), N-(5-(4-bromobenzylidene)-4-oxo-4,5-dihydrothiazol-2-yl) naphthalene-1-sulfonamide (Pitstop 2), solvates thereof, tautomer thereof, or pharmaceutically acceptable salts thereof.

In some embodiments, the agent is administered to the subject prior to and/or concurrently or simultaneously with the PSMA-targeted therapy.

In some embodiments, the PSMA-targeted therapy can include a PSMA-targeted radioligand. The PSMA-targeted radioligand can include, for example, a PSMA-targeting ligand or PSMA inhibitor selected from PSMA-11, PSMA-617, EB-PSMA-617, PSMA-R2, PSMA-1007, PSMA-914, PSMA-N064, PSMA-TTC, TX591, CTT1057, rhPSMA-7, rhPSMA-10, FSU-8802-PMPA, DCFPYL, DCFBC, ZJ24, RB11033, PSMA I&T, PSMA-1, CA008, CA009, CA011, MIP-1555, MIP-1519, MIP-1545, MIP-1427, MIP-1428, MIP-1379, MIP-1072, MIP-1095, MIP-1558, MIP-1405, MIP-1404, analogs thereof, or derivatives thereof.

In some embodiments, the PSMA-targeted radioligand can include a radiolabel selected from Thorium-227 (²²⁷Th), Actinium-225 (²²⁵AC), Astatine-211 (²¹¹At), Lead-203 (²⁰³Pb), Lead-212 (²¹²Pb), Terbium-149 (¹⁴⁹Tb), Terbium-161 (¹⁶¹Tb), and Lutetium-177 (¹⁷⁷Lu), Iodine-123 (¹²³I), Iodine-124 (¹²⁴I), Iodine-125 (¹²⁵I) or Iodine-131 (¹³¹I).

In other embodiments, the PSMA-targeted radioligand is selected from ²²⁵Ac-PSMA-617, ¹⁷⁷Lu-PSMA I&T, ¹⁷⁷Lu-PSMA-R2, ¹⁷⁷Lu-PSMA-617, ¹⁷⁷Lu-EB-PSMA-617, ¹⁷⁷Lu-rhPSMA-10.x, ²¹³Bi-PSMA-617, ²¹¹At-DCAtBzL, ¹²³I-MIP-1095, ¹²⁴I-MIP-1095, or ¹³¹I-MIP-1095.

In some embodiments, the non-cancer tissue can include salivary gland tissues and/or lacrimal gland tissues and the method can include administering to the subject a pharmacologically effective amount of an agent that inhibits PSMA-mediated cellular uptake and/or retention of the PSMA-targeted radioligand therapy by the non-cancer salivary gland tissues and/or lacrimal gland tissues in the subject.

In some embodiments, a pharmacologically effective amount of the agent administered to the subject can be an amount effective to reduce PSMA-targeted radioligand therapy induced salivary gland tissue damage of the subject and/or reduce PSMA-targeted radioligand therapy induced lacrimal gland tissue damage of the subject.

In some embodiments, the salivary gland damage can give rise to a side effect selected from xerostomia, thickened saliva, reduced saliva, mouth sores, hoarseness, trouble swallowing, loss of taste, or combinations thereof. A pharmacologically effective amount of the inhibitor of PSMA-mediated cellular internalization administered to the subject can be an amount effective to reduce, inhibit, treat, and/or prevent these side effects.

In some embodiments, the lacrimal gland damage can give rise to a side effect selected from xerophthalmia, dry eye syndrome (keratoconjunctivitis sicca), impaired tear secretion, visual disturbance, corneal ulceration, or combinations thereof. A pharmacologically effective amount of the inhibitor of PSMA-mediated cellular internalization administered to the subject can be an amount effective to reduce, inhibit, treat, and/or prevent these side effects.

In some embodiments, the subject is being treated for a PSMA expressing cancer. The PSMA expressing cancer can include, for example, prostate cancer, such as metastatic prostate cancer, as well as thyroid cancer, hepatocellular cancer, renal cancer, glioblastoma, breast cancer, lung cancer, gastric cancer, colorectal cancer, osteosarcoma, pancreatic cancer, ovarian, endometrial, and vulvar caner, head and/or neck cancers, and other cancers.

Other embodiments described herein relate to a method of treating PSMA expressing cancer in a subject in need thereof. The method can include administering to the subject a therapeutically effective amount of PSMA-targeted radioligand and a pharmacologically effective amount of an agent that inhibits PSMA-mediated cellular retention of the PSMA-targeted radioligand by non-cancer salivary gland tissues and/or lacrimal gland tissues in the subject.

In some embodiments, the agent includes an inhibitor of PSMA-mediated cellular internalization.

In some embodiments, the agent can compete for binding and/or uptake of the PSMA-targeted therapy to PSMA-expressing cancer tissue, PSMA-expressing non-cancerous tumors, and/or PSMA-expressing tumor vasculature in the subject.

In other embodiments, the agent does not compete for binding and/or uptake of the PSMA-targeted therapy to PSMA-expressing cancer tissue, PSMA-expressing non-cancerous tumors, and/or PSMA-expressing tumor vasculature in the subject.

In some embodiments, the agent comprises an inhibitor of clathrin mediated endocytosis. The inhibitor of CME can form a complex with a binding site on a terminal domain of clathrin heavy chain to interfere with CME.

In some embodiments, an inhibitor of CME that can form a complex with a binding site on a terminal domain of clathrin heavy chain can include an aquaporin (AQP) inhibitor, preferably an aquaglyceroporin inhibitor.

In some embodiments, the AQP inhibitor can include at least one of a 1-(5-nitrothiophene-3-yl)urea compound, a 1-(1H-indole-3-yl)urea compound, or a 4-(carbamoylamino)benzoate compound.

In other embodiments, the AQP inhibitor can include at least one of DFP00173, DFP00172, SEW00835, SEW00834, SEW00833, SEW00832, F2740-0111, Z4339273301, HTS13286, 9016645, 9053871, 7791389, or IVK/0050432.

In other embodiments, the inhibitor of CME can include 2-(4-aminobenzyl)-1,3-dioxo-2,3-dihydro-1H-benzo[de]isoquinoline-5-sulfonate (Pitstop 1), N-(5-(4-bromobenzylidene)-4-oxo-4,5-dihydrothiazol-2-yl) naphthalene-1-sulfonamide (Pitstop 2), solvates thereof, tautomer thereof, or pharmaceutically acceptable salts thereof.

In some embodiments, the PSMA-targeted radioligand can include, for example, a PSMA-targeting ligand or PSMA inhibitor selected from PSMA-11, PSMA-617, EB-PSMA-617, PSMA-R2, PSMA-1007, PSMA-914, PSMA-N064, PSMA-TTC, TX591, CTT1057, rhPSMA-7, rhPSMA-10, FSU-8802-PMPA, DCFPYL, DCFBC, ZJ24, RB11033, PSMA I&T, PSMA-1, CA008, CA009, CA011, MIP-1555, MIP-1519, MIP-1545, MIP-1427, MIP-1428, MIP-1379, MIP-1072, MIP-1095, MIP-1558, MIP-1405, MIP-1404, analogs thereof, or derivatives thereof.

In some embodiments, the PSMA-targeted radioligand can include a radiolabel selected from Thorium-227 (²²⁷Th), Actinium-225 (²²⁵AC), Astatine-211 (²¹¹At), Lead-203 (²⁰³Pb), Lead-212 (²¹²Pb), Terbium-149 (¹⁴⁹Tb), Terbium-161 (¹⁶¹Tb), and Lutetium-177 (¹⁷⁷Lu), Iodine-123 (¹²³I), Iodine-124 (¹²⁴I), Iodine-125 (¹²⁵I) or Iodine-131 (¹³¹I).

In other embodiments, the PSMA-targeted radioligand is selected from ²²⁵Ac-PSMA-617, ¹⁷⁷Lu-EB-PSMA-617, ¹⁷⁷Lu-rhPSMA-10.x, ¹⁷⁷Lu-PSMA I&T, ¹⁷⁷Lu-PSMA-R2, ¹⁷⁷Lu-PSMA-617, ²¹³Bi-PSMA-617, ²¹¹At-DCAtBzL, ¹²³I-MIP-1095, ¹²⁴I-MIP-1095, or ¹³¹I-MIP-1095.

In some embodiments, the PSMA expressing cancer can include prostate cancer, such as metastatic prostate cancer, as well as thyroid cancer, hepatocellular cancer, renal cancer, glioblastoma, breast cancer, lung cancer, gastric cancer, colorectal cancer, osteosarcoma, pancreatic cancer, ovarian, endometrial, and vulvar caner, head and/or neck cancers, and other cancers.

Still other embodiments described herein relate to a radioligand therapy for treating PSMA expressing cancers that includes a PSMA-targeted radioligand and an inhibitor of PSMA-targeted radioligand retention in PSMA expressing non-cancerous tissue.

In some embodiments, the inhibitor can compete for binding and/or uptake of the PSMA-targeted therapy to PSMA-expressing cancer tissue, PSMA-expressing non-cancerous tumors, and/or PSMA-expressing tumor vasculature in the subject.

In other embodiments, the inhibitor of PSMA mediated cellular internalization does not compete for binding and/or uptake of the PSMA-targeted therapy to a PSMA expressing cancer tissue, PSMA expressing non-cancerous tumor, and/or PSMA expressing tumor vasculature in the subject.

In some embodiments, the inhibitor of PSMA-mediated cellular internalization can be an inhibitor of clathrin mediated endocytosis. The inhibitor of CME can form a complex with a binding site on a terminal domain of clathrin heavy chain that interferes with CME.

In some embodiments, an inhibitor of CME that can form a complex with a binding site on a terminal domain of clathrin heavy chain can include an aquaporin (AQP) inhibitor, preferably an aquaglyceroporin inhibitor.

In some embodiments, the AQP inhibitor can include at least one of a 1-(5-nitrothiophene-3-yl)urea compound, a 1-(1H-indole-3-yl)urea compound, or a 4-(carbamoylamino)benzoate compound.

In other embodiments, the AQP inhibitor can include at least one of DFP00173, DFP00172, SEW00835, SEW00834, SEW00833, SEW00832, F2740-0111, Z4339273301, HTS13286, 9016645, 9053871, 7791389, or IVK/0050432.

In other embodiments, the inhibitor of CME can include 2-(4-aminobenzyl)-1,3-dioxo-2,3-dihydro-1H-benzo[de]isoquinoline-5-sulfonate (Pitstop 1), N-(5-(4-bromobenzylidene)-4-oxo-4,5-dihydrothiazol-2-yl) naphthalene-1-sulfonamide (Pitstop 2), solvates thereof, tautomer thereof, or pharmaceutically acceptable salts thereof.

In some embodiments, the PSMA-targeted radioligand can include, for example, a PSMA-targeting ligand or PSMA inhibitor selected from PSMA-11, PSMA-617, EB-PSMA-617, PSMA-R2, PSMA-1007, PSMA-914, PSMA-N064, PSMA-TTC, TX591, CTT1057, rhPSMA-7, rhPSMA-10, FSU-8802-PMPA, DCFPYL, DCFBC, ZJ24, RB11033, PSMA I&T, PSMA-1, CA008, CA009, CA011, MIP-1555, MIP-1519, MIP-1545, MIP-1427, MIP-1428, MIP-1379, MIP-1072, MIP-1095, MIP-1558, MIP-1405, MIP-1404, analogs, or derivatives thereof.

In some embodiments, the PSMA-targeted radioligand can include a radiolabel selected from Thorium-227 (²²⁷Th), Actinium-225 (²²⁵AC), Astatine-211 (²¹¹At), Lead-203 (²⁰³Pb), Lead-212 (²¹²Pb), Terbium-149 (¹⁴⁹Tb), Terbium-161 (¹⁶¹Tb), and Lutetium-177 (¹⁷⁷Lu), Iodine-123 (¹²³I), Iodine-124 (¹²⁴I), Iodine-125 (¹²⁵I) or Iodine-131 (¹³¹I).

In other embodiments, the PSMA-targeted radioligand is selected from ²²⁵Ac-PSMA-617, ¹⁷⁷Lu-EB-PSMA-617, ¹⁷⁷Lu-rhPSMA-10.x, ¹⁷⁷Lu-PSMA I&T, ¹⁷⁷Lu-PSMA-R2, ¹⁷⁷Lu-PSMA-617, ²¹³Bi-PSMA-617, ²¹¹At-DCAtBzL, ¹²³I-MIP-1095, ¹²⁴I-MIP-1095, or ¹³¹I-MIP-1095.

In some embodiments, the dosage of the inhibitor of PSMA-mediated cellular internalization administered to the subject is an amount effective to reduce PSMA-targeted radioligand therapy induced xerostomia in salivary gland tissues and/or xerophthalmia in lacrimal gland tissues of the subject.

Still other embodiments relate to a unit dosage form of a formulation of a PSMA-targeted radioligand and an inhibitor of PSMA-targeted radioligand retention in PSMA expressing non-cancer tissue. In some embodiments, the inhibitor can include a PSMA mediated cellular internalization inhibitor that does not compete for binding and/or uptake of the PSMA-targeted therapy to a PSMA expressing cancer tissue, PSMA expressing non-cancerous tumor, and/or PSMA expressing tumor vasculature in the subject.

In some embodiments, the unit dosage of the inhibitor is an amount effective to reduce PSMA-targeted radioligand therapy induced xerostomia in salivary gland tissues and/or xerophthalmia lacrimal gland tissue of the subject.

### BRIEF DESCRIPTION OF THE DRAWINGS

The following is a brief description of the drawings which are presented for the purpose of illustrating the invention and not for the purpose of limiting them.

FIG. 1 illustrates images showing the retention of PSMA-targeting positron emission tomography (PET) imaging ligands in mice bearing human prostate cancer xenograft. Mice were injected with clinically used [⁶⁸Ga]-PSMA-11, the right had AQP3-specific inhibitor, while the left shows high salivary uptake without blocking. The right panel showed comparable (PSMA-positive PC3pip) tumor uptake (on the flank).

FIG. 2 illustrates a graph showing region-based SUV (mean) from regions defined over muscle, salivary, kidney, and tumor of mice injected with clinically used [⁶⁸Ga]-PSMA-11 with and without administration of the AQP3-specific inhibitor.

FIGS. 3(A-B) illustrate graphs showing biodistribution of [¹⁷⁷Lu]PSMA-617 injected in mice implanted with PSMA-positive PC3pip tumor cells at 0.5 hour, 4 hours, 1 day, 3 days and 7 days post-injection.

FIGS. 4(A-B) illustrate graphs showing biodistribution of [¹⁷⁷Lu]PSMA-617 with DFP inhibition injected in mice implanted with PSMA-positive PC3pip tumor cells at 0.5 hour, 4 hours, 1 day, 3 days and 7 days post-injection.

FIGS. 5(A-B) illustrate graphs showing biodistribution of [¹⁷⁷Lu]PSMA-617 with PitStop2 inhibition injected in mice implanted with PSMA-positive PC3pip tumor cells at 0.5 hour, 4 hours, 1 day, 3 days and 7 days post-injection.

FIG. 6 illustrates a chart comparing area under curve (AUC) of accumulative activity of PSMA-617 in mice implanted with PSMA-positive PC3pip tumor cells injected with PSMA-617, PSMA-617+DFP inhibition, and PSMA-617+PitStop2 inhibition. The columns next to DFP or PIStop2 are the ratios between (DFP inhibition+PSMA-167)/(PSMA-617 alone) or (PitStop2 inhibition+PSMA-617)/(PSMA-617 alone).

FIGS. 7(A-C) illustrate schematic images showing molecular docking of DFP and PitStop2 to the clathrin terminal domain.

FIG. 8 illustrates a graph showing biodistribution of [¹⁷⁷Lu]PSMA-617 injected in mice implanted with PSMA-positive PC3pip tumor cells at 24 hours with different DFP inhibitions.

### DETAILED DESCRIPTION

All scientific and technical terms used in this application have meanings commonly used in the art unless otherwise specified. The definitions provided herein are to facilitate understanding of certain terms used frequently herein and are not meant to limit the scope of the application.

As used herein, the verb “comprise” as is used in this description and in the claims and its conjugations are used in its non-limiting sense to mean that items following the word are included, but items not specifically mentioned are not excluded. The present invention may suitably “comprise”, “consist of”, or “consist essentially of”, the steps, elements, and/or reagents described in the claims.

It is further noted that the claims may be drafted to exclude any optional element. As such, this statement is intended to serve as antecedent basis for use of such exclusive terminology as “solely”, “only” and the like in connection with the recitation of claim elements, or the use of a “negative” limitation.

The term “or” as used herein should be understood to mean “and/or”, unless the context clearly indicates otherwise.

As used herein, the term “targeting moiety” or “targeting ligand” can refer to a molecule or molecules that are able to bind to and complex with a biomarker. The term can also refer to a functional group that serves to target or direct a compound described herein to a particular location, cell type, diseased tissue, or association. In general, a “targeting moiety” can be directed against a biomarker.

As used herein, the term “neoplastic disorder” can refer to a disease state in a subject in which there are cells and/or tissues which proliferate abnormally. Neoplastic disorders can include, but are not limited to, cancers, sarcomas, tumors, leukemias, lymphomas, and the like.

The terms “cancer” or “tumor” refer to any neoplastic growth in a subject, including an initial tumor and any metastases. The cancer can be of the liquid or solid tumor type. Liquid tumors include tumors of hematological origin, including, e.g., myelomas (e.g., multiple myeloma), leukemias (e.g., Waldenstrom's syndrome, chronic lymphocytic leukemia, other leukemias), and lymphomas (e.g., B-cell lymphomas, non-Hodgkin's lymphoma). Solid tumors can originate in organs and include cancers of the lungs, brain, breasts, prostate, ovaries, colon, kidneys and liver.

The terms “cancer cell” or “tumor cell” can refer to cells that divide at an abnormal (i.e., increased) rate. Cancer cells include, but are not limited to, carcinomas, such as squamous cell carcinoma, non-small cell carcinoma (e.g., non-small cell lung carcinoma), small cell carcinoma (e.g., small cell lung carcinoma), basal cell carcinoma, sweat gland carcinoma, sebaceous gland carcinoma, adenocarcinoma, papillary carcinoma, papillary adenocarcinoma, cystadenocarcinoma, medullary carcinoma, undifferentiated carcinoma, bronchogenic carcinoma, melanoma, renal cell carcinoma, hepatoma-liver cell carcinoma, bile duct carcinoma, cholangiocarcinoma, papillary carcinoma, transitional cell carcinoma, choriocarcinoma, semonoma, embryonal carcinoma, mammary carcinomas, gastrointestinal carcinoma, colonic carcinomas, bladder carcinoma, prostate carcinoma, and squamous cell carcinoma of the neck and head region; sarcomas, such as fibrosarcoma, myxosarcoma, liposarcoma, chondrosarcoma, osteogenic sarcoma, chordosarcoma, angiosarcoma, endotheliosarcoma, lymphangiosarcoma, synoviosarcoma and mesotheliosarcoma; hematologic cancers, such as myelomas, leukemias (e.g., acute myelogenous leukemia, chronic lymphocytic leukemia, granulocytic leukemia, monocytic leukemia, lymphocytic leukemia), lymphomas (e.g., follicular lymphoma, mantle cell lymphoma, diffuse large B-cell lymphoma, malignant lymphoma, plasmocytoma, reticulum cell sarcoma, or Hodgkin's disease), and tumors of the nervous system including glioma, glioblastoma multiform, meningoma, medulloblastoma, schwannoma and epidymoma.

The phrases “parenteral administration” and “administered parenterally” are art-recognized terms, and include modes of administration other than enteral and topical administration, such as injections, and include, without limitation, intravenous, intramuscular, intrapleural, intravascular, intrapericardial, intraarterial, intrathecal, intracapsular, intraorbital, intracardiac, intradermal, intraperitoneal, transtracheal, subcutaneous, subcuticular, intra-articular, subcapsular, subarachnoid, intraspinal and intrastemal injection and infusion.

The phrases “systemic administration,” “administered systemically,” “peripheral administration” and “administered peripherally” as used herein mean the administration of a compound, agent or other material other than directly into a specific tissue, organ, or region of the subject being treated, such that it enters the animal's system and, thus, is subject to metabolism and other like processes, for example, subcutaneous administration.

A “therapeutically effective amount” of a compound with respect to the subject method of treatment, refers to an amount of the compound(s) in a preparation which, when administered as part of a desired dosage regimen (to a mammal, preferably a human) alleviates a symptom, ameliorates a condition, or slows the onset of disease conditions according to clinically acceptable standards for the disorder or condition to be treated or the cosmetic purpose, e.g., at a reasonable benefit/risk ratio applicable to any medical treatment.

As used herein, the term “treating” can include reversing, alleviating, inhibiting the progression of, preventing or reducing the likelihood of the disease, disorder, or condition to which such term applies, or one or more symptoms or manifestations of such disease, disorder or condition. Preventing refers to causing a disease, disorder, condition, or symptom or manifestation of such, or worsening of the severity of such, not to occur. Accordingly, the presently disclosed compounds can be administered prophylactically to prevent or reduce the incidence or recurrence of the disease, disorder, or condition.

The terms “therapeutic agent”, “drug”, “medicament” and “bioactive substance” are art-recognized and include molecules and other agents that are biologically, physiologically, or pharmacologically active substances that act locally or systemically in a patient or subject to treat a disease or condition. The terms include without limitation pharmaceutically acceptable salts thereof and prodrugs. Such agents may be acidic, basic, or salts; they may be neutral molecules, polar molecules, or molecular complexes capable of hydrogen bonding; they may be prodrugs in the form of ethers, esters, amides and the like that are biologically activated when administered into a patient or subject.

“PSMA” refers to Prostate Specific Membrane Antigen, a potential carcinoma marker that has been hypothesized to serve as a target for imaging and cytotoxic treatment modalities for cancer.

As used herein, the term “subject” can refer to any animal, including, but not limited to, humans and non-human animals (e.g., rodents, arthropods, insects, fish (e.g., zebrafish)), non-human primates, ovines, bovines, ruminants, lagomorphs, porcines, caprines, equines, canines, felines, aves, etc.), which is to be the recipient of a particular treatment.

Throughout the description, where compositions are described as having, including, or comprising, specific components, it is contemplated that compositions also consist essentially of, or consist of, the recited components. Similarly, where methods or processes are described as having, including, or comprising specific process steps, the processes also consist essentially of, or consist of, the recited processing steps. Further, it should be understood that the order of steps or order for performing certain actions is immaterial so long as the compositions and methods described herein remains operable. Moreover, two or more steps or actions can be conducted simultaneously.

Embodiments described herein relate to methods of inhibiting retention of a prostate-specific membrane antigen (PSMA)-targeted therapy in PSMA expressing non-cancer tissue of a subject, and particularly relates to methods of reducing PSMA-targeted radioligand therapy induced salivary gland and/or lacrimal gland damage in a subject receiving PSMA-targeted radioligand therapy for cancer.

It has been shown that during systemic therapy targeting PSMA overexpressing cancer or cancerous tissue in a subject collateral damage of normal tissue from a PSMA-targeted therapy, such as the uptake of a radioligand agent in the salivary glands and lacrimal glands, can occur. We found that PSMA-ligand retention in salivary glands and lacrimal glands can be associated with an enhanced clathrin mediated endocytosis (CME) of PSMA and the retention of radioligands in the salivary glands can be reduced by interfering with and/or inhibiting CME. We identified and tested chemical compounds, such as aquaglyceroporin (AQP3)-specific inhibitors as well as other known CME inhibitors, in mouse models of human prostate cancer (PC3pip) xenograft administered PSMA-targeted radioligands and found an at least 50% reduction in salivary accumulation of clinically used PSMA-targeted imaging radioligands without substantial reduction in tumor uptake. Thus, administration of an agent that inhibits PSMA-mediated cellular retention of the PSMA-targeted therapy by the non-cancer tissue can be used clinically to improve the specificity and reduce the toxicity of PSMA-targeted therapies.

Accordingly, in some embodiments, a method of inhibiting retention of PSMA-targeted therapy in PSMA expressing non-cancer tissue of a subject can include administering to a subject being treated with the PSMA-targeted therapy a pharmacologically effective amount of an agent that inhibits PSMA-mediated cellular retention of the PSMA-targeted therapy by the non-cancer tissue. By PSMA-expressing non-cancer tissue it is meant normal or healthy organ or tissue expressing PSMA that is not cancer, cancerous tissue, nor PSMA-expressing vasculature (or neovasculature) that supports cancer or cancerous tissue. In some embodiments, the PSMA-expressing non-cancer tissue is selected from kidney, lacrimal gland tissues, salivary gland tissues, and the small intestine tissues.

The agent that inhibits PSMA-mediated cellular retention of the PSMA-targeted therapy by the non-cancer tissue can include any agent capable of inhibiting, and/or reducing PSMA internalization, accumulation, and/or retention of a PSMA-targeted therapy described herein in the PSMA-expressing non-cancer tissue.

In some embodiments, the agent or inhibitor can compete for binding and/or uptake of the PSMA-targeted therapy to PSMA-expressing cancer tissue, PSMA-expressing non-cancerous tumors, and/or PSMA-expressing tumor vasculature in the subject.

In other embodiments, such an agent or inhibitor of PSMA-mediated cellular internalization does not compete for binding and/or uptake of the PSMA-targeted therapy to PSMA-expressing cancer tissue, PSMA-expressing non-cancerous tumors, and/or PSMA-expressing tumor vasculature in the subject.

In some embodiments, the agent can include an agent developed for inhibiting CME. By an agent developed for inhibiting CME or inhibitor of CME, it is meant an agent that interacts with clathrin expressed by a cell to at least partially inhibit the functional activity of clathrin as may be determined by a reduction in CME in the cell. The inhibition of CME can be a total or partial inhibition in the functional activity of clathrin.

In some embodiments, the inhibitor of CME can include an agent that forms a complex with a binding site on a terminal domain (TD) of clathrin heavy chain and interferes with CME. By terminal domain of clathrin heavy chain, it is meant the N-terminal β-propeller region of clathrin heavy chain 1, such as defined by amino acids 2-479 of Genbank Accession No. NM-004859.3, and fragments thereof, providing the binding site for an inhibitor of CME.

Methods for screening inhibitors of CME in accordance with embodiments described herein are well known to a person skilled in the art. Computer programs and systems for this are commercially available, and any conventionally known such methodology and/or modelling software can be utilized (e.g., AutoDock 4.2, AutoDock Vina (The Scripps Research Institute, La Jolla, CA, USA). Whilst the screening or modelling of inhibitors of CME and their interaction with the clathrin terminal domain binding site may at least in part be accomplished manually with graphical representations and 3-dimensional modelling, computer based systems are the preferred option. Such computer assisted modelling can involve the provision of an initial structural representation of an inhibitor and electronically modelling it into a representation of the clathrin TD binding site, such as an electron density map of the binding site to determine whether the fit of the putative inhibitor forms the desired complex with the binding site or whether optimization of the structure of the putative inhibitor is desirable. The designing of an inhibitor of CME may also involve modelling the inhibitor to at least partially fit or conform to the structure of a pharmacophore or other clathrin inhibitor as described herein.

The design and optimization of the structural features of the putative inhibitor can involve modification of the backbone scaffold of the inhibitor and/or modification (e.g., replacement), exclusion or addition of scaffold substituents to alter the interaction of the inhibitor with the binding site. This can take into account steric, lipophilic, and/or charge considerations (e.g., attraction and/or repulsion) to provide for formation of a complex with the clathrin TD in accordance with embodiments described herein and/or to alter (i.e., increase or decrease) the affinity with which the inhibitor binds to the clathrin TD binding site. The design and modelling of the inhibitor can also take into account the formation of hydrogen bonds between the inhibitor and the clathrin TD wherein the hydrogen bond(s) can be formed directly between the inhibitor and the clathrin TD or be mediated by water, or be a mixture of those two possibilities. The design of compounds and modelling of ligand binding is for instance described in United States Patent Application Publication Nos. 2004/0219653, US 2009/0275047, US 2005/0170431 and US 2010/0247569, all of which are herein incorporated by reference in their entirety.

Further, the crystal structure of a 55 kD fragment of the terminal domain (TD) of the heavy chain of rat clathrin consisting of amino acid residues 1-494 and the methodology for obtaining same has previously been reported, as has the crystal structure for a fragment comprising amino acid residues 1-363 of the terminal domain TD of clathrin heavy chain respectively complexed with a peptide derived from β-arrestin 2 or the β-subunit of the AP-3 complex (the crystal structural data for which is available under ID codes 1C9L and 1C9I from the RBSC Protein Data Bank, Rutgers, the State University of New Jersey, Taylor Road, Piscataway, NJ, USA) (ter Haar, 1998; ter Haar, 2000), reference to which can also be had for the design and provision of an inhibitor of CME, and the contents of all of which are incorporated herein by reference in their entirety.

In some embodiments, the inhibitor of CME can include a compound having formula (I), formula (V), or a pharmaceutically acceptable salt thereof as disclosed in WO 2013/010218, which is incorporated herein by reference in its entirety.

A compound of formula (I) includes:

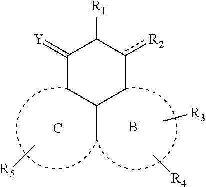

wherein:
R₁ is alkyl, alkenyl, alkylaryl, aryl, or a ring having from 5 to 7 ring atoms including from 0 to 3 heteroatoms typically selected from O, N or S, wherein the alkyl, alkenyl, alkylaryl, aryl, and ring group are optionally substituted; and
R₂ is O, S or NH; or
R₁, X, and R₂ form a ring A, the ring having from 5 to 7 ring atoms including from 0 to 3 heteroatoms typically selected from O, N or S and being optionally substituted; and
X is N or a carbon atom;
Y is O, S, or NH;
ring B and ring C each independently have 5 to 7 ring atoms including from 0 to 3 heteroatoms typically selected from O, N or S; and
R₃, R₄, and R₅ are each independently H or an optional substituent.

A compound of formula (V) includes:

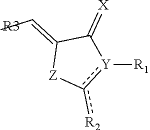

wherein:
X is O, S, or NH;
Y is O, N or a carbon atom;
Z is O, S or NH;
R₁ is H, CO₂H, SO₃H, PO₄H, alkyl, alkenyl, alkylcarboxy, or alkylaryl;
R₂ is O, S, NH, NHR′, NHS(═O)₂R′, S(═O)₂R′, sulfonyl, NHC(═O)NH, NHC(═S)NH, or NHC(═O);
R′ is H, alkyl, alkenyl, alkylaryl, aryl, the alkyl, alkenyl, alkylaryl and aryl group being optionally substituted; and
R₃ is aryl or a polycyclic group having at least 2 fused rings each independently having from 5 to 7 ring atoms, the aryl and the polycyclic group being optionally substituted.

In some embodiments, a compound of formula (I) or formula (V) can include 2-(4-aminobenzyl)-1,3-dioxo-2,3-dihydro-1H-benzo[de]isoquinoline-5-sulfonate (Pitstop 1), N-(5-(4-bromobenzylidene)-4-oxo-4,5-dihydrothiazol-2-yl) naphthalene-1-sulfonamide (Pitstop 2), solvates thereof, tautomer thereof, or pharmaceutically acceptable salts thereof.

Other examples of inhibitors for CME that can potentially inhibit PSMA-mediated cellular internalization include methyl-β-cyclodextrin (β-CD), hydrophobic amines (e.g., phenothiazines, monodansylcadaverine and chloroquine), monensin, hyperosmotic sucrose and dynasore. Phenothiazines include, but are not limited to, chlorpromazine, fluphenazine, mesoridazine, perphenazine, prochlorperazine, promazine, thioridazine, trifluoperazine and triflupromazine. β-CD inhibits CME by selectively removing cholesterol from the plasma membrane. Hydrophobic amines inhibit CME by affecting the function of clathrin and clathrin-coated vesicles. Monensin inhibits CME by dissipating a proton gradient. Hyperosmotic sucrose inhibits CME by preventing clathrin and adaptors from interacting. Dynasore inhibits dynamin GTPase which facilitates the formation of coated pits.

Still other examples of agents that form a complex with a binding site on a terminal domain of clathrin heavy chain and can inhibit PSMA-mediated cellular internalization include agents initially developed for inhibiting aquaporin (AQP), such as aquaglyceroporin inhibitors.

Aquaporins (AQPs) are a family of transmembrane protein channels accounting for transcellular water permeability. Aquaporins are found in virtually all life forms, from bacteria to plants to animals. In humans, they are found in cells throughout the body. In addition to being permeable to water, some AQPs can be permeable to small solutes, including cations and glycerol, and gases. Many aquaporins form tetramers as their functional configuration. Based on their structure and permeability characteristics, AQPs are subdivided into classical AQPs, primarily permeable to water but also to ions and gases (AQP0, AQP1, AQP2, AQP4, AQP5, AQP6, AQP8); aquaporins permeable to glycerol and other solutes in addition to water (AQP3, AQP7, AQP9, AQP10); and non-classical AQPs of uncertain permeability to water and/or glycerol (AQP11, AQP12).

Aquaporin inhibiting agents for use in a method described herein can therefore include AQP0, AQP1, AQP2, AQP3, AQP4, AQP5, AQP6, AQP7, AQP8, AQP9, AQP10, AQP11, and/or AQP12 inhibiting agents. Aquaporin inhibiting agents can include selective AQP inhibitors or pan-aquaporin inhibitors.

In certain embodiments, agents that form a complex with a binding site on a terminal domain of clathrin heavy chain and can inhibit PSMA-mediated cellular internalization are aquaglyceroporin inhibitors, such as AQP3 inhibitors and/or AQP7 inhibitors. AQP3 and/or AQP7 inhibitors for use in a method described herein can include, for example, DFP00173, DFP00172, SEW00835, SEW00834, SEW00833, SEW00832, F2740-0111, Z4339273301, HTS13286, 9016645, 9053871, 7791389, or IVK/0050432.

In some embodiments, the AQP3 inhibitor can include at least one of a 1-(5-nitrothiophene-3-yl)urea compound, a 1-(1H-indole-3-yl)urea compound, or a 4-(carbamoylamino)benzoate compound. For example, the AQP3 inhibitor can be the potent and selective AQP3 inhibitor, DFP00173. DFP00173 is capable of inhibiting mouse and human AQP3 with an IC50 of ~0.1-0.4 μM. (Sonntag Y, et al. Identification and characterization of potent and selective aquaporin-3 and aquaporin-7 inhibitors. J Biol Chem. 2019 May 3; 294 (18): 7377-7387).

Other AQP inhibitors that can that form a complex with a binding site on a terminal domain of clathrin heavy chain and can inhibit PSMA-mediated cellular internalization are AQP9 inhibitors. AQP9 shares a high amino acid sequence similarity with AQP3 and is highly expressed in the lacrimal glands. Accordingly, in certain embodiments, the inhibitor of PSMA-mediated cellular internalization can be an AQP9 inhibitor. AQP9 inhibitors for use in a method described herein can include, for example, HTS13286, phloretin, CD05595, RF03176, HTS13772, analogs and derivatives thereof.

In other embodiments, the AQP inhibitor can include at least one of DFP00173, DFP00172, SEW00835, SEW00834, SEW00833, SEW00832, F2740-0111, Z4339273301, HTS13286, 9016645, 9053871, 7791389, or IVK/0050432.

Additional inhibitors of PSMA-mediated cellular retention of a PSMA-targeted therapy by the non-cancer tissue can be identified via in silico analysis. In some embodiments, inhibiting agents can be identified using in silico molecular reverse docking modeling and molecular dynamics (MD) simulation analysis. For example, Induced Fit Docking-Molecular Dynamics (IFD-MD) analysis can be used to generate candidate agent and target binding models for evaluation along with free energy perturbation+ (FEP+) calculations to challenge the generated models. IFD-MD/FEP+predictions can then be integrated into an active learning scheme to further assist in evaluating candidate inhibiting agents for use in a method described herein.

In some embodiments, in vivo mouse model PET imaging assays can be used to screen candidate inhibiting agents for the ability to block internalization and/or retention of a PSMA-targeted PET imaging ligand, such as [⁶⁸Ga]PSMA-11 and protect PSMA expressing non-cancer tissue, such as salivary and/or lacrimal tissue, in a subject from tissue damage during PSMA-targeted radioligand therapy. For example, a candidate inhibitor of PSMA-mediated cellular internalization can be administered to a mouse model in combination with a [¹⁷⁷Lu]PSMA-617 theranostic radioligand to assess a candidate inhibitor's ability to mitigate salivary tissue damage from the radioligand.

Methods described herein include treating PSMA expressing cancer in a subject in need thereof by administering a therapeutically effective amount of a PSMA-targeted therapy and a pharmacologically effective amount of an inhibitor PSMA-mediated cellular internalization to the subject. Pathological studies indicate that PSMA is expressed by virtually all prostate cancers, and its expression is further increased in poorly differentiated, metastatic, and hormone-refractory carcinomas. Higher PSMA expression is also found in cancer cells from castration-resistant prostate cancer patients. Increased PSMA expression is reported to correlate with the risk of early prostate cancer recurrence after radical prostatectomy. In addition to being overexpressed in prostate cancer (PCa), PSMA is also expressed in the neovasculature of neoplasms including but not limited to conventional (clear cell) renal carcinoma, transitional cell carcinoma of the urinary bladder, testicular embryonal carcinoma, colonic adenocarcinoma, neuroendocrine carcinoma, glioblastoma multiforme, malignant melanoma, pancreatic ductal carcinoma, non-small cell lung carcinoma, soft tissue carcinoma, breast carcinoma, and prostatic adenocarcinoma.

In any of the methods described herein, the PSMA-targeting moiety of a PSMA-targeted therapy may have recognition for a particular PSMA expressing cancer, cancer tissue, or other tissue-related material associated with a particular PSMA expressing cancer. In particular embodiments, the PSMA targeting moiety of a PSMA-targeted therapy can include any molecule, or complex of molecules, which is/are capable of interacting with the cell surface PSMA glycoprotein biomarker of a PSMA expressing cancer cell. The PSMA-targeting moiety can interact with the PSMA glycoprotein cell surface biomarker through non-covalent binding, covalent binding, hydrogen binding, van der Waals forces, ionic bonds, hydrophobic interactions, electrostatic interaction, and/or combinations thereof.

The PSMA targeting moiety can include, but is not limited to, synthetic compounds, natural compounds or products, macromolecular entities, bioengineered molecules (e.g., polypeptides, lipids, polynucleotides, antibodies, antibody fragments), and small entities (e.g., small molecules, neurotransmitters, substrates, ligands, hormones and elemental compounds). By way of example, the PSMA-targeting moiety can comprise a PSMA ligand. In exemplary embodiments, the PSMA-targeting moiety can comprise a highly negatively charged PSMA ligand.

In some embodiments, a PSMA-targeting moiety of a PSMA-targeted therapy described herein can include a PSMA-targeting ligand or PSMA inhibitor selected from PSMA-11, PSMA-617, EB-PSMA-617, PSMA-R2, PSMA-1007, PSMA-914, PSMA-N064, PSMA-TTC, TX591, CTT1057, rhPSMA-7, rhPSMA-10, FSU-8802-PMPA, DCFPYL, DCFBC, ZJ24, RB11033, PSMA I&T, PSMA-1, CA008, CA009, CA011, analogs, or derivatives thereof.

In other embodiments, the PSMA-targeting ligand can include a Glu-Urea based PSMA ligand. Such compounds include those described in U.S. Pat. Nos. 8,211,401; 8,211,402; 8,465,725; 8,487,129; and 8,562, 945; and in PCT/US2014/011047. In some embodiments, the compound is MIP-1555, MIP-1519, MIP-1545, MIP-1427, MIP-1428, MIP-1379, MIP-1072, MIP-1095, MIP-1558, MIP-1405, MIP-1404, analogs, and derivatives thereof.

In some embodiments, the PSMA-targeted therapy includes a PSMA-targeted radioligand therapy. PSMA-targeted radioligand therapy may also be referred to as peptide-receptor radionuclide therapy (PRRT), systemic radiation, targeted radionuclide therapy (TRT), targeted radiotherapy or molecular radiotherapy. A PSMA-targeted radioligand for use in any of the methods described herein can combine a PSMA-targeting moiety that binds to PSMA expressed by cancer cells and a radioactive isotope, causing DNA damage that inhibits tumor growth and replication. PSMA-targeting allows for delivery of radiation to the tumor, while limiting damage to the surrounding normal tissue. PSMA-targeted radioligand therapy is typically a systemic treatment, reaching cells throughout the body by travelling through the bloodstream.

The ionizing radiation emitted by radionuclides conjugated to the PSMA-targeting ligand can kill cancer cells by damaging their DNA, causing the tumors to shrink. The biological action of a radiopharmaceutical is determined by the form of ionizing radiation emitted by the radionuclide. While imaging procedures in nuclear medicine require radionuclides that will emit γ (gamma) radiation able to penetrate the body, a different class of radionuclides possessing optimal relative biological effectiveness is needed for radionuclide therapy. The radionuclides best suited for tumor therapy are those emitting ionizing radiation with short penetration into the tissue, such as a (alpha) or β (beta) emitters, which release their energy in the proximity of their targets.

In any of the methods described herein, the radionuclide may be, but is not limited to, a radioactive isotope of Ga, Y, Tc, In, I, Tb, Lu, Re, Pb, At, Bi, Ac, or Th. Illustrative radionuclides include, but are not limited to Thorium-227 (²²⁷Th), Actinium-225 (²²⁵AC), Astatine-211 (211 At), Lead-203 (²⁰³Pb), Lead-212 (²¹²Pb), Terbium-149 (¹⁴⁹Tb), Terbium-161 (¹⁶¹Tb), and Lutetium-177 (¹⁷⁷Lu), Iodine-123 (¹²³I), Iodine-124 (¹²⁴I), Iodine-125 (¹²⁵I) or Iodine-131 (¹³¹I). In certain embodiments the radionuclide is selected from ²²⁵Ac and ¹⁷⁷Lu.

Examples of a PSMA-targeted radioligand agent is selected from the group consisting of ²²⁵Ac-PSMA-617, 177Lu-PSMA I&T, ¹⁷⁷Lu-PSMA-R2, 177Lu-PSMA-617, 177Lu-EB-PSMA-617, 177Lu-rhPSMA-10.x, ²¹³Bi-PSMA-617, ²¹¹At-DCAtBzL, ¹²³I-MIP-1095, ¹²⁴I-MIP-1095, or ¹³¹I-MIP-1095.

In other embodiments, the PSMA-targeted therapy can include a PSMA ligand-anticancer agent conjugate. For example, the PSMA ligand-anticancer agent conjugate can include a PSMA-targeted antibody-drug conjugate (PSMA ADC). PSMA ADCs can include a humanized or human monoclonal PSMA-targeted antibody conjugated to cytotoxic small molecule compounds through chemical linkers.

In particular embodiments, the cytotoxic small molecule compound can include derivatives of calicheamicin, a class of highly cytotoxic enediyne antibiotics which kill cells by causing DNA double-strand breaks, analogs thereof and/or derivatives of the tubulin polymerization inhibitors, dolastatin 10 (auristatins) and maytansine. In some embodiments, the cytotoxic small molecule is an maytansine analog selected from maytansinoid DM1 (mertansine) and maytansinoid DM4 (ravtansine). In particular embodiments, the anticancer agent is an auristatin derivative, selected from the group consisting of monomethylauristatin norephedrine (MMAE), monomethylauristatin phenylalanine (MMAF), analogs, and derivatives thereof.

Linkers for use in a PSMA ADC are designed to be stable in the blood stream (to conform to the increased circulation time of mAbs) and labile at the cancer site to allow efficient release of the cytotoxic compounds once the PSMA ADC is taken up by the cancer cells. PSMA ADC linkers can be characterized as either cleavable, or as non-cleavable. In some embodiments, the linker can include a lysosomally cleavable dipeptide, such as valine-citrulline (vc or Val-Cit). In particular embodiments, the PSMA-ligand-drug conjugate can include VcMMAE (MC-Val-Cit-PAB-MMAE).

One of ordinary skill in the art would recognize that other radiolabeled PSMA-targeted radioligands and/or PSMA ADCs, indeed any PSMA-targeted therapeutic cancer agent known in the art that may be internalized or retained by PSMA-expressing non-cancer tissue would be suitable for use with the presently disclosed methods.

In any of the above methods or treatment methods, unless otherwise specified, the inhibitor of PSMA-mediated cellular internalization and/or a PSMA-targeted radiotherapy can be provided in a pharmaceutical composition formulated for systemic, topical and/or localized administration.

Pharmaceutical compositions include sterile aqueous solutions suitable for injection and sterile powders for the extemporaneous preparation of injectable solutions. Such injectable compositions will be fluid to the extent that syringability exists. Injectable solutions will typically be prepared by incorporating the active(s) in the selected carrier prior to sterilizing the solution by filtration. In the case of sterile powders, preferred methods of preparation are vacuum drying and freeze-drying techniques which yield a powder of the active and any additional desired ingredient from previously sterile filtered solutions thereof. As will be understood, a pharmaceutical composition as described herein can be provided in a form wherein the components of the composition are admixed with one another.

Alternatively, in another embodiment, the inhibitor of PSMA-mediated cellular internalization and/or PSMA-targeted therapy can be provided partially or totally separately for combination with other components to form the composition, such as in the form of a kit.

For oral administration, the inhibitor of PSMA-mediated cellular internalization can be formulated into any orally acceptable carrier deemed suitable. In particular, the inhibitor can be formulated with an inert diluent, an assimilable edible carrier or it may be enclosed in a hard or soft shell gelatin capsule. Moreover, an inhibitor of PSMA-mediated cellular internalization can be provided in the form of ingestible tablets, buccal tablets, troches, capsules, elixirs, suspensions or syrups. Inhibitors of PSMA-mediated cellular internalization as described herein can also be formulated into topically acceptable preparations including creams, lotions or ointments for internal or external application. Topically acceptable compositions can be applied directly to the site of treatment including by way of dressings and the like impregnated with the preparation.

A pharmaceutical composition can also incorporate one or more preservatives such as parabens, chlorobutanol, phenol, and sorbic acid. In addition, prolonged absorption of the composition may be brought about by the inclusion of agents for delaying absorption such as aluminium monosterate. Tablets, troches; pills, capsules and like can also contain one or more of the following: a binder such as gum tragacanth, acacia, corn starch or gelatin, a disintegrating agent such as corn starch, potato starch or alginic acid, a lubricant such as magnesium stearate a sweetening agent such as sucrose, lactose or saccharin; and a flavoring agent.

Pharmaceutically acceptable carriers include any suitable conventionally known physiologically acceptable solvents, dispersion media, isotonic preparations and solutions including for instance, physiological saline. Use of such ingredients and media for pharmaceutically active substances is well known. It is particularly preferred to formulate compositions in unit dosage form for ease of administration and uniformity of dosage. The inhibitors of PSMA-mediated cellular internalization as described herein can also be provided in unit dosage form. A unit dosage form as used herein is to be taken to mean physically discrete units, each containing a predetermined quantity of the selected compound(s) calculated to produce a therapeutic or prophylactic effect. When the unit dosage form is a capsule, it can contain the active in a liquid carrier. Various other ingredients may be present as coatings or to otherwise modify the physical form of the unit dosage. For instance, tablets, pills or capsules may be coated with shellac, sugars or both.

Systemic administration includes delivery of an aqueous solution, preferably a buffered aqueous solution, including an inhibitor of PSMA-mediated cellular internalization. Systemic formulations typically also include a dispersant. Systemic administration is typically done parenterally (e.g., intravenously or intramuscularly). However, systemic administration can also be carried out by oral administration. By way of example, pharmaceutical compositions including an inhibitor of PSMA-mediated cellular internalization and/or a PSMA-targeted radiotherapy described herein can be intravenously administered to a subject that is known to or suspected of having a PSMA expressing tumor.

Pharmaceutical compositions including agents described herein can be formulated to allow delivery in sufficient amounts and for a period of time(s) to be therapeutically effective. Single or multiple administrations of an agent(s) can be given. Agents described herein can be administered to a subject in therapeutically effective quantity.

In any of the above methods, the inhibitor of PSMA-mediated cellular internalization may be administered to the subject in an amount effective to prevent, reduce, and/or inhibit PSMA-mediated cellular accumulation and/or retention of a PSMA-targeted therapy by the non-cancer tissue. In certain embodiments, the inhibiting agent may be administered to the subject in an amount that is effective to inhibit PSMA-mediated cellular retention of a PSMA-targeted therapy by the non-cancer tissue without inhibiting the PSMA-targeted therapy from binding to PSMA expressing cancerous tissue.

In some embodiments, a pharmacologically effective amount of the inhibitor of PSMA-mediated cellular internalization administered to the subject is an amount effective to inhibit PSMA-mediated cellular retention of a PSMA-targeted therapy by the non-cancer tissue by at least 1%, 5%, 10%, 20%, 30%, 40%, 50%, 60%, 70%, 80%, 90% compared to a control subject administered PSMA-targeted therapy without the administration of the inhibitor of PSMA-mediated cellular internalization.

In other embodiments, a pharmacologically effective amount of the inhibitor of PSMA-mediated cellular internalization administered to the subject is an amount effective to reduce PSMA-targeted radioligand therapy induced salivary gland tissue damage of the subject and/or reduce PSMA-targeted radioligand therapy induced lacrimal gland tissues damage of the subject.

Salivary damage can give rise to a side effect selected from xerostomia, thickened saliva, reduced saliva, mouth sores, hoarseness, trouble swallowing, loss of taste, or combinations thereof. A pharmacologically effective amount of the inhibitor of PSMA-mediated cellular internalization administered to the subject can be an amount effective to reduce, inhibit, treat, and/or prevent the side effect. Xerostomia is characterized by dry mouth resulting from reduced or absent saliva flow. Thus, in some embodiments, the pharmacologically effective amount of the inhibitor of PSMA-mediated cellular internalization can be an amount effective to reduce PSMA-targeted radioligand therapy induced xerostomia in salivary gland tissues of the subject.

Lacrimal gland damage can give rise to a side effect selected from xerophthalmia, dry eye syndrome (keratoconjunctivitis sicca), impaired tear secretion, visual disturbance, corneal ulceration, or combinations thereof. A pharmacologically effective amount of the inhibitor of PSMA-mediated cellular internalization administered to the subject is an amount effective to reduce, inhibit, treat, and/or prevent the side effect. Xerophthalmia is characterized by dryness of the conjunctiva and cornea due to a failure of the secretory activity of the mucin-secreting goblet cells of the conjunctiva. In some cases, xerophthalmia can spread to the cornea causing dry eye syndrome (keratoconjunctivitis sicca) and, if severe, keratomalacia. Thus, in some embodiments, the pharmacologically effective amount of the inhibitor of PSMA-mediated cellular internalization can be an amount effective to reduce PSMA-targeted radioligand therapy induced xerophthalmia in lacrimal gland tissues of the subject.

In any of the above methods or treatment methods, unless otherwise specified, the inhibitor of PSMA-mediated cellular internalization and/or PSMA-targeted therapy may be administered to the subject from about 0.01 mg/kg to about 100 mg/kg, such as 0.01, 0.05, 0.09, 0.1, 0.5, 0.9, 1.0, 1.1, 1.5, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 45, 50, 60, 70, 80, 90 or 100 mg/kg, per day. Dosage forms (composition) suitable for internal administration generally contain from about 0.01 milligram to about 500 milligrams of active ingredient per unit. In some embodiments, the amount is from about 0.05 mg/kg to about 50 mg/kg. In yet other embodiments, the amount is from about 1 mg/kg to about 50 mg/kg. In some embodiments, the amount is from about 0.05 mg/kg to about 75 mg/kg. In some embodiments, the amount is from about 0.05 mg/kg to about 50 mg/kg. In some embodiments, the amount is from about 0.05 mg/kg to about 25 mg/kg. In some embodiments, the amount is from about 0.05 mg/kg to about 10 mg/kg. In some embodiments, the amount is from about 0.05 mg/kg to about 5 mg/kg. In some embodiments, the amount is from about 1 mg/kg to about 40 mg/kg. In some embodiments, the amount is from about 1 mg/kg to about 30 mg/kg. In some embodiments, the amount is from about 1 mg/kg to about 20 mg/kg. In some embodiments, the amount is from about 1 mg/kg to about 10 mg/kg. In some embodiments, the amount is from about 10 mg/kg to about 40 mg/kg. In some embodiments, the amount is from about 10 mg/kg to about 30 mg/kg. In some embodiments, the amount is from about 10 mg/kg to about 20 mg/kg.

The amount of the inhibitor of PSMA-mediated cellular internalization and/or PSMA-targeted therapy administered to the subject can depend on the characteristics of the subject, such as general health, age, sex, body weight and tolerance to drugs as well as the degree, severity and type of rejection. The skilled artisan will be able to determine appropriate dosages depending on these and other factors using standard clinical techniques.

In addition, in vitro or in vivo assays can be employed to identify desired dosage ranges. The dose to be employed can also depend on the route of administration, the seriousness of the disease, and the subject's circumstances. Effective doses may be extrapolated from dose-response curves derived from in vitro or animal model test systems. The amount of the inhibitor of PSMA-mediated cellular internalization and/or PSMA-targeted therapy can also depend on the cancer state or condition being treated along with the clinical factors and the route of administration of the compound.

In some embodiments, the inhibitor of PSMA-mediated cellular internalization is administered in combination with the PSMA-targeted therapy, for example, in a method of treating a PSMA expressing cancer in a subject in need thereof. By “in combination with” is meant the administration of the inhibitor of PSMA-mediated cellular internalization with a PSMA-targeted therapy either before, simultaneously sequentially, or a combination thereof. Therefore, a cell, a tissue, or a subject can receive the inhibitor and the PSMA-targeted therapy at the same time (i.e., simultaneously) or at different times (i.e., sequentially, in either order, i.e., before or after, on the same day or on different days), so long as the effect of the combination of both agents is achieved in the cell, tissue, or the subject. When administered sequentially, the agents can be administered within about 1, 5, 10, 30, 60, 120, 180, 240 minutes or longer of one another. In other embodiments, agents administered sequentially, can be administered within 1, 5, 10, 15, 20 or more days of one another. Where the inhibitor of PSMA-mediated cellular internalization and PSMA-targeted therapy are administered simultaneously, they can be administered to the cell or administered to the subject as separate pharmaceutical compositions, each including either one or more presently disclosed inhibitors of PSMA-mediated cellular internalization or one or more PSMA-targeted therapies described herein, or they can contact the cell or tissue as a single composition or be administered to a subject as a single pharmaceutical composition including both agents.

In particular embodiments, the inhibitor of PSMA-mediated cellular internalization is administered to the subject prior to the PSMA-targeted therapy being administered. In such embodiments, the subject is “pre-treated” with the inhibitor of PSMA-mediated cellular internalization to improve specificity and reduce the toxicity of PSMA-targeted therapy. In other embodiments, the inhibitor of PSMA-mediated cellular internalization is administered to the subject simultaneously with the PSMA-targeted therapy.

In some embodiments, the inhibitor of PSMA-mediated cellular internalization and PSMA-targeted therapy are administered for about 1 to about 20 treatment cycles. In some embodiments, the inhibitor and PSMA-targeted therapy and are administered from about 5 to about 15 treatment cycles. In some embodiments, the PSMA-targeted therapy is a PSMA-targeted radioligand administered in a cumulative amount from about 0.1 GBq to about 100 GBq. In particular embodiments, the PSMA-targeted radioligand is administered in a cumulative amount from about 50 GBq to about 75 GBq.

In some embodiments, the subject being treated with a PSMA-targeted therapy has not received previous treatment for cancer. In some embodiments, the subject is being treated for a PSMA expressing cancer. The PSMA expressing cancer can include prostate cancer, such as metastatic prostate cancer, as well as thyroid cancer, hepatocellular cancer, renal cancer, glioblastoma, breast cancer, lung cancer, gastric cancer, colorectal cancer, osteosarcoma, pancreatic cancer, ovarian, endometrial, and vulvar caner, head and/or neck cancers, and other cancers. In certain embodiments, the cancer is prostate cancer or metastatic prostate cancer. In some embodiments, the prostate cancer is castration-resistant metastatic prostate cancer. In some embodiments, the subject treated by the methods provided herein, may be a subject that has had prior chemotherapy with at least one taxane, such as docetaxel, cabazitaxel, and combinations thereof. In some embodiments, the subject may also be one that has had prior treatment with one or more antiandrogens, such as enzalutamide, abiraterone, or combinations thereof. In some embodiments the subject was previously treated with at least one taxane and at least one antiandrogen prior to treatment with a method described herein.

The specific process utilized to administer one or more of the therapeutic agents in the methods of the present invention when used to inhibit PSMA-mediated cellular internalization of a PSMA-targeted therapy to the non-cancer tissue in a subject are more particularly described below in the following examples. The following examples are for the purpose of illustration only and is not intended to limit the scope of the claims, which are appended hereto.

### Examples

In this Example, we describe a novel approach to reduce the retention of radioligands in the salivary glands by interfering with clathrin-mediated endocytosis (CME) for PSMA internalization. We tested and identified chemical compounds such as DFP00173 (DFP for short), which is an aquaglyceroporin (AQP3)-specific inhibitor designed to inhibit glycerol permeability of this water channel that also transports glycerol in the salivary glands. We applied DFP along with other known CME inhibitors into the mouse models of prostate cancer (PC3pip) xenograft.

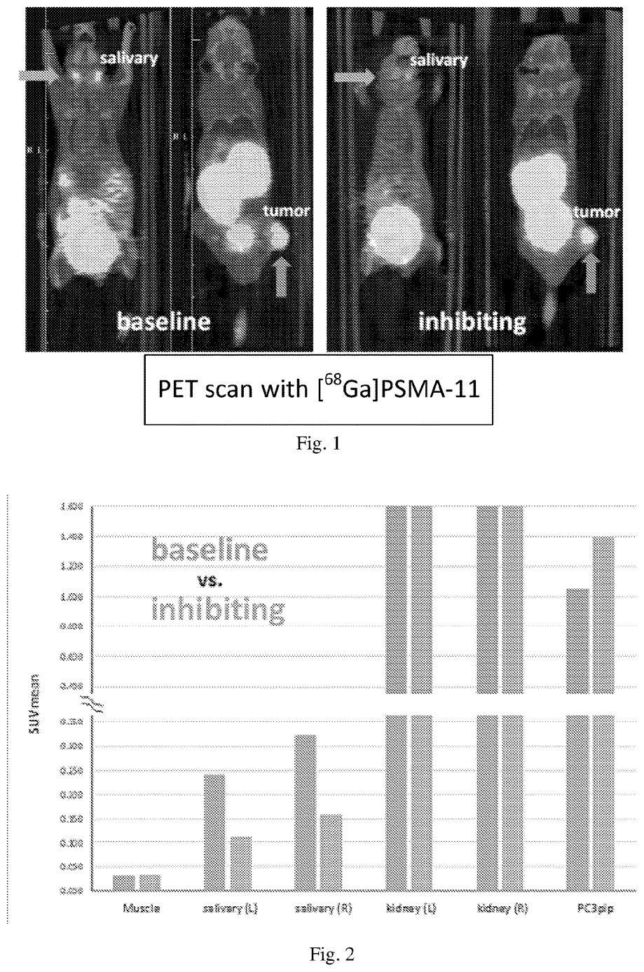

FIG. 1 illustrates images showing the uptake of PSMA-ligand in salivary glands was reduced by half with DFP during PET imaging. The images show coronal cuts of PET/CT overlays from microPET scans using [⁶⁸Ga]PSMA-11, baseline (left) and salivary inhibition with DFP00173 (right). Adult thymic mice (8 weeks old) were implanted with PSMA-positive PC3pip tumor cells to grow on the flank, and injected i.v. via tail vein with 200 μCi (7.4 MBq) of [⁶⁸Ga]PSMA-11 and imaged one-hour post-injection. For inhibition, DFP (0.05 μg/kg in 100 μL saline solution) was injected s.c. 5 minutes before [⁶⁸Ga]PSMA-11 injection. The orange arrows point to parotid gland uptake, and red arrows to PC3pip tumor uptake.

FIG. 2 illustrates a graph showing region-based SUV (mean) from regions defined over muscle, salivary, kidney, and tumor of mice injected with clinically used [⁶⁸Ga]-PSMA-11 with and without administration of the AQP3-specific inhibitor.

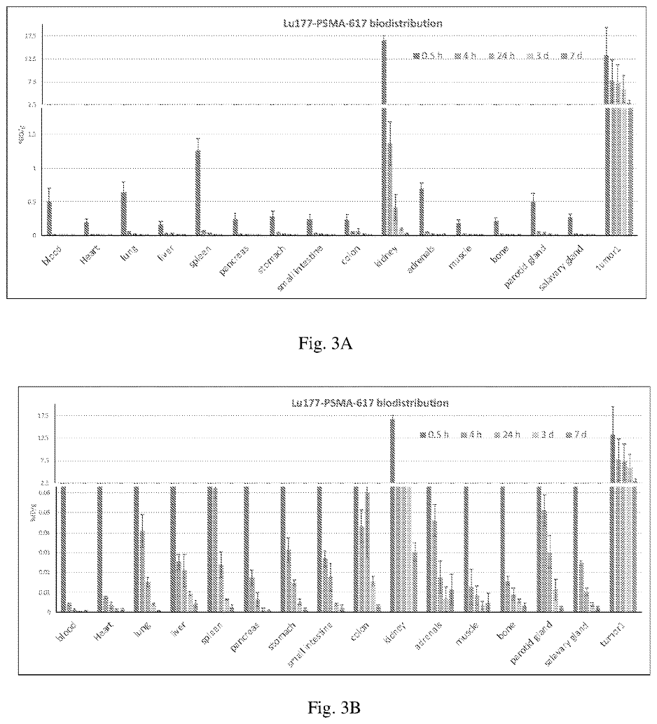

FIG. 3(A-B) illustrate graphs showing biodistribution of injected [¹⁷⁷Lu]PSMA-617 in mice implanted with PSMA-positive PC3pip tumor cells at 0.5 hour, 4 hours, 1 day, 3 days and 7 days post-injection. Adult thymic mice (8 weeks old) were implanted with PSMA-positive PC3pip tumor cells to grow on the flank, injected i.v. via tail vein with 75 μCi (7.4 MBq) of [¹⁷⁷Lu]PSMA-617 and euthanized in group of 3 (n=3) at 0.5 hour, 4 hours, 1 day, 3 days and 7 days post-injection. The tissues and organs were harvested, weighted and counted, and expressed as % ID/g. (the lower panel with a finer scale)

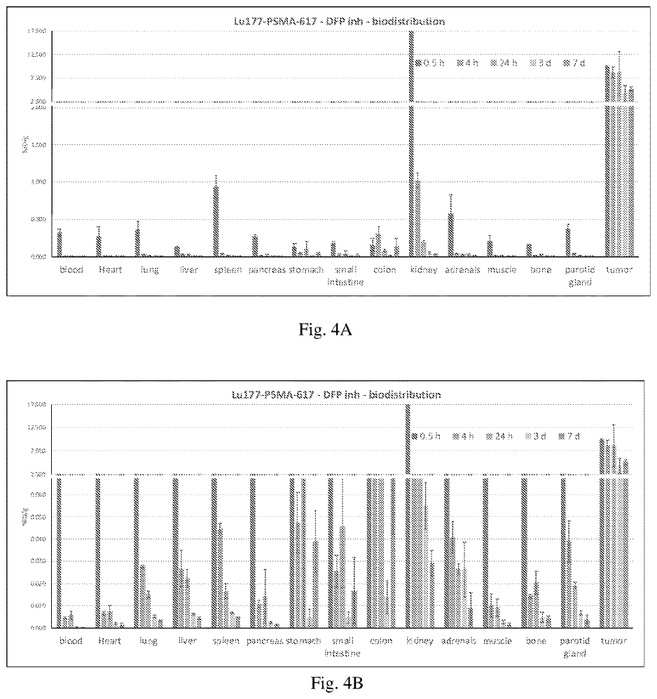

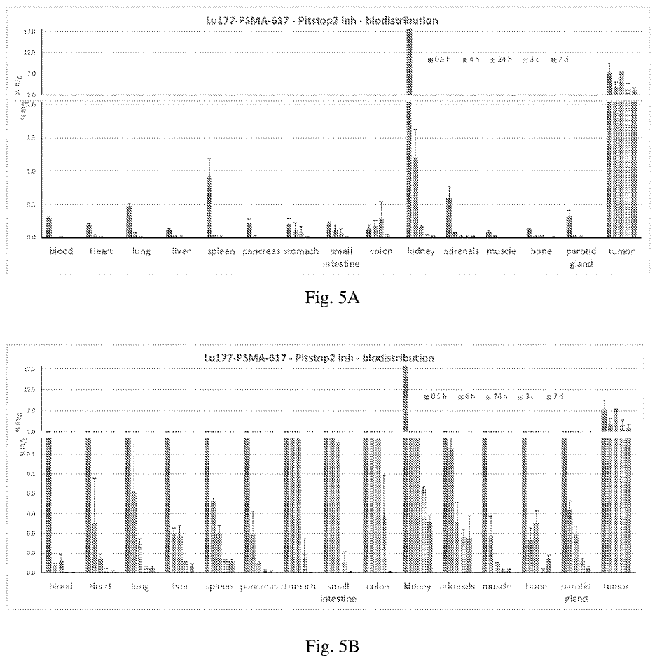

FIG. 4(A-B) illustrate graphs showing biodistribution of injected [¹⁷⁷Lu]PSMA-617 with DFP inhibition in mice implanted with PSMA-positive PC3pip tumor cells at 0.5 hour, 4 hours, 1 day, 3 days and 7 days post-injection. Adult thymic mice (8 weeks old) were implanted with PSMA-positive PC3pip tumor cells to grow on the flank, injected i.v. via tail vein with 75 μCi (7.4 MBq) of [¹⁷⁷Lu]PSMA-617 and euthanized in group of 3 (n=3) at 0.5 hour, 4 hours, 1 day, 3 days and 7 days post-injection. The tissues and organs were harvested, weighted and counted, and expressed as % ID/g. For inhibition, DFP (0.05 μg/kg in 100 μL saline solution) was injected s.c. 5 minutes before [¹⁷⁷Lu]PSMA-617 injection. (the lower panel with a finer scale).

FIG. 5(A-B) illustrate graphs showing biodistribution of injected [¹⁷⁷Lu]PSMA-617 with PitStop2 inhibition in mice implanted with PSMA-positive PC3pip tumor cells at 0.5 hour, 4 hours, 1 day, 3 days and 7 days post-injection. Adult thymic mice (8 weeks old) were implanted with PSMA-positive PC3pip tumor cells to grow on the flank, injected i.v. via tail vein with 75 μCi (7.4 MBq) of [¹⁷⁷Lu]PSMA-617 and euthanized in group of 3 (n=3) at 0.5 hour, 4 hours, 1 day, 3 days and 7 days post-injection. The tissues and organs were harvested, weighted and counted, and expressed as % ID/g. For inhibition, Pitstop2 (20 μg/kg in a mixed solution of DMSO and corn oil) was injected i.p. one hour before [¹⁷⁷Lu]PSMA-617 injection. (the lower panel with a finer scale).

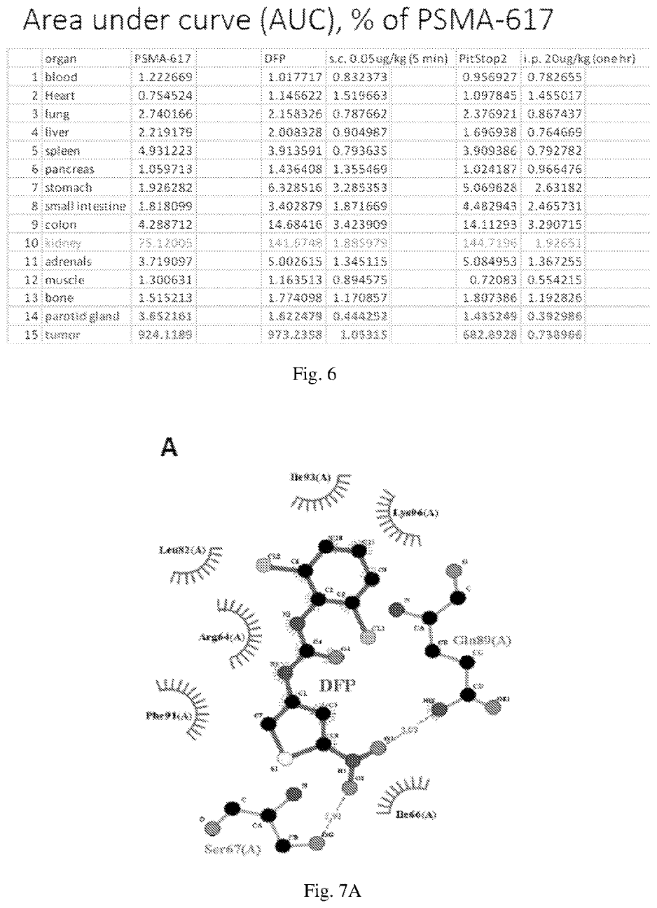

FIG. 6 illustrates a chart comparing area under curve (AUC) of accumulative activity of PSMA-617 in mice implanted with PSMA-positive PC3pip tumor cells injected with PSMA-617, PSMA-617+DFP inhibition, and PSMA-617+PitStop2 inhibition. The columns next to DFP or PIStop2 are the ratios between (DFP inhibition+PSMA-167)/(PSMA-617 alone) or (PitStop2 inhibition+PSMA-617)/(PSMA-617 alone). Area under the curves (AUCs) were calculated using trapezoidal approximation. For example, DFP inhibition brought down accumulative dose in the parotid salivary glands to 44.4% of that of PSMA-617 applied alone while maintaining the tumor dose at 105%.

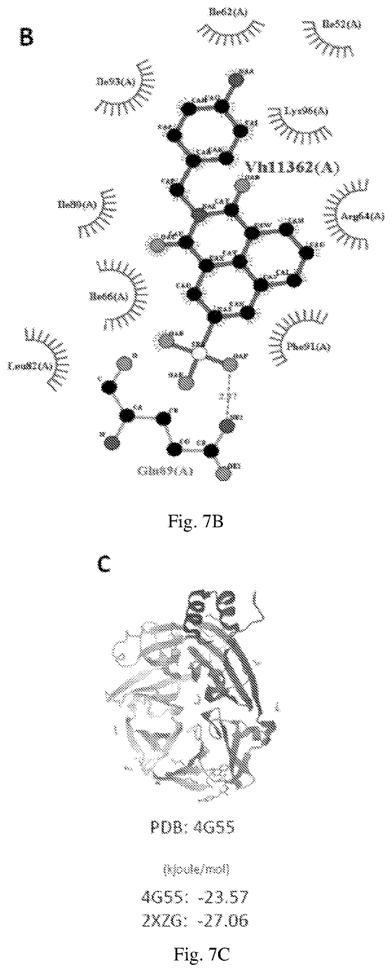

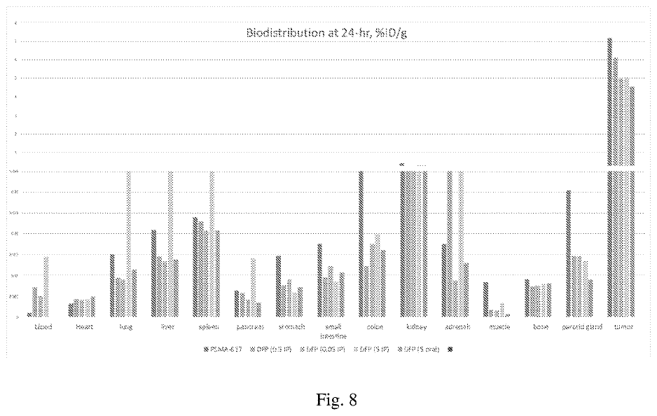

FIG. 7(A-C) illustrates schematic images showing molecular docking of DFP and PitStop2 to the clathrin terminal domain. A) Molecular docking of DFP to clathrin terminal domain (clathrin heavy chain, CHC); B) Similar docking of PitStop2 to CHC; C) Calculated binding free energy of DFP to CHC converted to a >30 μM affinity between DFP and CHC, while the experimentally determined binding affinity between PitStop and CHC is 12~18 μM. Protein structures of CHC (4G55 and 2XZG) were downloaded from PDB.

FIG. 8 illustrates a graph showing biodistribution of injected [¹⁷⁷Lu]PSMA-617 in mice implanted with PSMA-positive PC3pip tumor cells at 24 hours with different DFP inhibitions. Adult thymic mice (8 weeks old) were implanted with PSMA-positive PC3pip tumor cells to grow on the flank, injected i.v. via tail vein with 75 μCi (7.4 MBq) of [¹⁷⁷Lu]PSMA-617 and euthanized in group of 2 (n=2) at 24 hours (1 day) post-injection. The tissues and organs were harvested, weighted and counted, and expressed as % ID/g (error bars are not shown for clarity of the display). For inhibition, DFP (0.05 IP) is 0.05 μg/kg in corn oil and 10% DMSO solution injected i.p. one hour before [¹⁷⁷Lu]PSMA-617 injection; DFP (0.5 IP): 0.5 μg/kg in corn oil and DMSO solution injected i.p. one hour before [¹⁷⁷Lu]PSMA-617 injection; DFP (5 IP): 5 μg/kg in corn oil and DMSO solution injected i.p. one hour before [¹⁷⁷Lu]PSMA-617 injection; DFP (5 oral): 5 g/kg in corn oil and DMSO solution gavaged orally 2.5 hour before [¹⁷⁷Lu]PSMA-617 injection.

FIGS. 3-6 and 8 show that administration of an inhibitor of CME can result in >50% reduction in salivary accumulation of clinically used [¹⁷⁷Lu]PSMA-617 without much reduction in tumor uptake. Interfering the internalization of PSMA-targeting radioligands through CME inhibition will protect the salivary glands (rate-limiting organ) and increase the therapeutic index of targeted radioligand therapy in humans. Although FDA approved [¹⁷⁷Lu]PSMA-617, the response duration for many late-stage prostate cancer patients was modest. By applying inhibitors of PSMA-mediated internalization to reduce salivary retention, dose escalation is expected to greatly enhance such breakthrough treatment for prostate cancer with a much better outcome. Preventing PSMA-ligand retention in the salivary glands by inhibiting CME is the key for developing new strategies to reduce the severity of targeted treatment-induced xerostomia. Without xerostomia, which relegates this to tertiary treatment, radioligand therapy can be used as a primary (first-line) treatment.

From the above description of the invention, those skilled in the art will perceive improvements, changes and modifications. Such improvements, changes and modifications within the skill of the art are intended to be covered by the appended claims. All references, publications, and patents cited in the present application are herein incorporated by reference in their entirety.

1. A method of inhibiting accumulation and/or retention of a prostate-specific membrane antigen (PSMA)-targeted therapy in PSMA expressing non-cancer tissue of a subject, the method comprising:
administering to a subject being treated with the PSMA-targeted therapy a pharmacologically effective amount of an agent that inhibits PSMA-mediated cellular retention of the PSMA-targeted therapy by the non-cancer tissue, wherein the agent comprises an inhibitor of clathrin mediated endocytosis, an aquaporin (AQP) inhibitor, or an aquaglyceroporin inhibitor.

2. (canceled)

3. The method of claim 1, wherein the agent does not compete for binding and/or uptake of the PSMA-targeted therapy to PSMA-expressing cancer tissue, PSMA-expressing non-cancerous tumors, and/or PSMA-expressing tumor vasculature in the subject.

4. (canceled)

5. The method of claim 1, wherein the inhibitor of clathrin mediated endocytosis forms a complex with a binding site on a terminal domain of clathrin heavy chain.

6. The method of claim 1, wherein the agent comprises the aquaporin (AQP) inhibitor or the aquaglyceroporin inhibitor.

7. The method of claim 6, wherein the AQP inhibitor comprises at least one of a 1-(5-nitrothiophene-3-yl)urea compound, a 1-(1H-indole-3-yl)urea compound, or a 4-(carbamoylamino)benzoate compound.

8. The method of claim 6, wherein the AQP inhibitor comprises at least one of DFP00173, DFP00172, SEW00835, SEW00834, SEW00833, SEW00832, F2740-0111, Z4339273301, HTS13286, 9016645, 9053871, 7791389, or IVK/0050432.

9. The method of claim 1, wherein the inhibitor of clathrin mediated endocytosis comprises 2-(4-aminobenzyl)-1,3-dioxo-2,3-dihydro-1H-benzo[de]isoquinoline-5-sulfonate (Pitstop 1), N-(5-(4-bromobenzylidene)-4-oxo-4,5-dihydrothiazol-2-yl) naphthalene-1-sulfonamide (Pitstop 2), solvates thereof, tautomer thereof, or pharmaceutically acceptable salts thereof.

10. The method of claim 1, wherein the agent is administered to the subject prior to the subject being treated with the PSMA-targeted therapy.

11. The method of claim 1, wherein the agent is administered to the subject simultaneously with the PSMA-targeted therapy.

12. The method of claim 1, wherein the non-cancer tissue comprises salivary gland tissues and/or lacrimal gland tissues.

13. The method of claim 1, wherein the PSMA-targeted therapy comprises a PSMA-targeted radioligand.

14. The method of claim 1, wherein the pharmacologically effective amount is an amount effective to reduce PSMA-targeted radioligand therapy induced xerostomia (dry mouth) in salivary gland tissues of the subject.

15. The method of claim 1, wherein the pharmacologically effective amount is an amount effective to reduce PSMA-targeted radioligand therapy induced xerophthalmia (dry eye) in lacrimal gland tissues of the subject.

16. The method of claim 13, wherein the PSMA-targeted radioligand comprising a PSMA-targeting ligand or PSMA inhibitor selected from PSMA-11, PSMA-617, EB-PSMA-617, PSMA-R2, PSMA-1007, PSMA-914, PSMA-N064, PSMA-TTC, TX591, CTT1057, rhPSMA-7, rhPSMA-10, FSU-8802-PMPA, DCFPYL, DCFBC, ZJ24, RB11033, PSMA I&T, PSMA-1, CA008, CA009, CA011, MIP-1555, MIP-1519, MIP-1545, MIP-1427, MIP-1428, MIP-1379, MIP-1072, MIP-1095, MIP-1558, MIP-1405, MIP-1404, analogs, or derivatives thereof.

17. The method of claim 13, wherein the PSMA-targeted radioligand comprises a radiolabel selected from Thorium-227 (²²⁷Th), Actinium-225 (²²⁵AC), Astatine-211 (²¹¹At), Lead-203 (²⁰³Pb), Lead-212 (²¹²Pb), Terbium-149 (¹⁴⁹Tb), Terbium-161 (¹⁶¹Tb), and Lutetium-177 (¹⁷⁷Lu), Iodine-123 (¹²³I), Iodine-124 (¹²⁴I), Iodine-125 (¹²⁵I) or Iodine-131 (¹³¹I).

18. The method of claim 13, wherein the PSMA-targeted radioligand is selected from ²²⁵Ac-PSMA-617, ¹⁷⁷Lu-PSMA I&T, ¹⁷⁷Lu-PSMA-R2, ¹⁷⁷Lu-PSMA-617, ¹⁷⁷Lu-EB-PSMA-617, ¹⁷⁷Lu-rhPSMA-10.x, ²¹³Bi-PSMA-617, ²¹¹At-DCAtBzL, ¹²³I-MIP-1095, ¹²⁴I-MIP-1095, or ¹³¹I-MIP-1095.

19. The method of claim 1, wherein the subject is being treated for a PSMA expressing cancer.

20. The method of claim 19, wherein the PSMA expressing cancer comprises metastatic prostate cancer.

21. A method of reducing PSMA-targeted radioligand therapy induced salivary gland and/or lacrimal gland damage in a subject receiving PSMA-targeted radioligand therapy for cancer, the method comprising:
administering to the subject a pharmacologically effective amount of an agent that inhibits PSMA-mediated cellular uptake and/or retention of the PSMA-targeted radioligand therapy by the non-cancer salivary gland tissues and/or lacrimal gland tissues in the subject, wherein the agent comprises an inhibitor of clathrin mediated endocytosis, an aquaporin (AQP) inhibitor, or an aquaglyceroporin inhibitor.

22-37. (canceled)

38. A method of treating PSMA expressing cancer in a subject in need thereof, the method comprising:
administering to the subject a therapeutically effective amount of PSMA-targeted radioligand and a therapeutically effective amount of an agent that inhibits PSMA-mediated cellular retention of the PSMA-targeted radioligand by non-cancer salivary gland tissues and/or lacrimal gland tissues in the subject, wherein the agent comprises an inhibitor of clathrin mediated endocytosis, an aquaporin (AQP) inhibitor, or an aquaglyceroporin inhibitor.

39-76. (canceled)
